(19) United States
(12) Patent Application Publication
(10) Pub. No.: US 2005/0130912 A1
(43) Pub. Date: Jun. 16, 2005

(54) Compositions for the improvement of obesity

(76) Inventors: Hyun-Woo Park, Kyunggi-do (KR); Sun-Young Kim, Kyunggi-do (KR); Wan-Gi Kim, Kyunggi-do (KR); Su-Jong Kim, Kyunggi-do (KR); Ji-Hyun Kim, Kyunggi-do (KR); Tae-Ryong Lee, Kyunggi-do (KR); Young-Chul Sim, Kyunggi-do (KR); Sang-Jun Lee, Kyunggi-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/507,779

(22) PCT Filed: Oct. 21, 2003
(86) PCT No.: PCT/KR03/02202

(30) Foreign Application Priority Data
Mar. 25, 2003 (KR) 10-2003-0018559

(51) Int. Cl. A61K 31/7048; A61K 31/353; A61K 31/198

(52) U.S. Cl. 514/27; 514/456; 514/565; 424/439

(57) ABSTRACT

The present invention relates to a composition containing genistein that accelerates the expression of the Carnitine Palmitoyl Transferase-1 (CPT-1) for improving or treating obesity. More particularly, the composition of the present invention comprises genistein that accelerates the expression of the CPT-1, an important enzyme for the lipolysis of fatty acid, and comprises carnitine that transports fatty acid into mitochondria and accelerates oxidation of fat in the oxidation process of fatty acid.

### FIELD OF THE INVENTION

The present invention relates to a composition containing genistein that accelerates the expression of the Carnitine Palmitoyl Transferase-1 (hereinafter, we call ‘CPT-1’) for improving or treating obesity. More particularly, the composition of the present invention comprises genistein that accelerates the expression of the CPT-1, an important enzyme for the lipolysis of fatty acid, and comprises carnitine that transports fatty acid into mitochondria and accelerates oxidation of fat in the oxidation process of fatty acid.

### BACKGROUND OF THE INVENTION

Obesity is a state of metabolic disorder caused by unbalance of intake and consumption of energy, resulting extra energy not consumed is accumulated as fat and therefore the fat in a body abnormally increases. Obesity is an important health problem throughout western and eastern countries, and it is reported that about 30˜40% of people have the problem of obesity. Obesity is a cause of stress, and an important reason for inducing hypertension, hyperlipemia, arteriosclerosis, heart disease, diabetes, or the like.

There are a lot of causes of obesity such as high-fatty and caloric food, insufficient exercise, disorders of endocrine system or pathologic problems. In addition, genetic factors are important, which can be estimated from fact that obesity frequently occurs in a certain family connected hereditary factors. It is reported that genetic factors affect to the generation of obesity at least 30˜50%.

Methods or therapies for treating and preventing obesity have been studied and researched widely throughout the world. Present methods comprise diet therapy decreasing food-intake to reduce energy, exercise therapy increasing energy consumption, surgical therapies such as cutting off parts of inner organs or suction of fat, and drug therapies using metabolic accelerator, appetite-suppressant or digest/absorption-suppressant. However, no satisfactory method has been reported and the above methods have side effects such as yo-yo effect that obesity becomes more serious after stopping the therapy; unbalanced nutrition condition due to diet therapy; or infections due to decreased immunity. In particular, drug therapy causes depression, insomnia, indigestion, or the like. Therefore it is strongly desired to invent a safe and effective method for treating and preventing obesity without side effects.

Considering various diseases caused by obesity, decrease of body fat is more important than simple reduction of body weight. Therefore, it is more desirable to seek a method to reduce accumulation of fat in the body and to accelerate oxidation of fat. Under this, a method for accelerating beta-oxidation of fat would be a main target of treating obesity. Among them, acceleration of oxidation of fatty acid can be achieved by controlling the expression of CPT-1, an enzyme determining the reactivity of beta-oxidation of fatty acid. Until now, a method for accelerating the expression of CPT-1 in order to increase oxidation of fat for the purpose of suppressing obesity has not been studied widely (McCarty, Medical Hypotheses 57(3): 324-336, 2001).

### SUMMARY OF THE INVENTION

The present inventors researched to find natural product that accelerates the expression of CPT-1, a rate-liming enzyme for oxidation of fatty acid, and found that genistein, a kind of soy bean isoflavone, can accelerate the expression of CPT-1 without side effects when administered orally and can reduce obesity, and finally completed the invention.

In addition, the present inventors found that when L-carnitine, which acts an important role in the transfer of fatty acid into mitochondria, is added to a composition containing genistein, synergic effect for accelerating the expression of CPT-1 can be obtained.

Therefore, an object of the present invention is to provide a composition for treating obesity that can accelerate the expression of CPT-1, a rate-liming enzyme for oxidation of fat, and as a result accelerates the oxidative metabolism of body fat.

### DETAILED DESCRIPTION OF THE INVENTION

The present invention provides a composition comprising genistein, a kind of soybean isoflavone, which accelerates the expression of Carnitine Palmitoyl Transferase-1 (CPT-1), an important enzyme in the lipolysis of fatty acid, and comprising L-carnitine, which acts an important role in the transfer of fatty acid into mitochondria and accelerates oxidation of fat, for treating obesity.

Hereinafter, the present invention is described in detail.

Obesity is resulted from accumulation of fat due to unbalance of energies between taken and consumed nutrition, disorders of metabolism including low internal secretion of fat-oxidation enzymes, low secretion of leptin-a kind of fat oxidation enzyme, and problems of adrenalin receptor and due to genetic factors.

Fatty acid is a component of fat and is transformed in a cell through beta-oxidation, TCA cycle and oxidative phosphorylation to a form to be use as energy consuming a lot of oxygen and generating ATP. However fatty acid can not pass through the membrane of mitochondria because of its big molecular size. These long chain fatty acids transferred into cytosol from blood can not pass through the membrane of mitochondria directly, but permeates into mitochondria by the following three (3) steps of enzymatic reactions:
1. A long chain fatty acid in the cytosol forms thiol ester with coenzyme A between carboxyl group of the fatty acid and thiol group of the coenzyme A (Co-A) by acyl-CoA synthetase existing outer membrane of the mitochondria. The above-formed fatty acyl-CoA has high energy compound properties like acetyl-CoA.
2. The fatty acyl-CoA ester can not pass through inner membrane of mitochondria. In order to transfer the fatty acid into the inside of mitochondria, CPT-1 existing on the outer surface of the inner membrane catalyzes transesterification of fatty acyl group with carnitine in Co-A. The above formed fatty acyl-carnitine ester passes through acyl-carnitine/carnitine transporter into the matrix of mitochonria by facilitated diffusion.
3. The fatty acyl-camitine is catalyzed by carnitine acyltransferase II to form fatty acyl-CoA.

Fatty acids transferred into the matrix of mitochondria through the above three steps of enzymatic reactions are transformed to acetyl Co-A by beta-oxidation, which finally transformed to electron and CO₂ by citric acid cycle. The above electron generates ATP through respiratory chain process (Lehninger et al., Principles of Biochemistry: 479-505, 1993).

The genistein of the present invention is a main component accelerating the expression of CPT-1 to promote the oxidation of fat, and represented by the following formula 1.

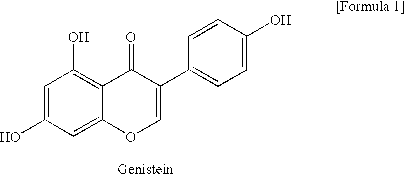

A genistein is a kind of isoflavone contained in, for example, soy bean, and has diphenolic ring as a chemical backbone. Isoflavones existing as a form of glycoside are transformed to an aglycone form such as genistein or daidzein by internal glucosidase. Isoflavone of soybean has similar structure and function with estrogen and therefore reported as phytoestrogen, and has various physiological effects such as ameliorating menopausal disorder (Albertazzi et al., Obstet Gynecol 91 (1): 6-11,1998, Anderson et al., Public Health Nutr 2(4): 489-504, 1999), remedying osteoporosis (Scheiber et al., Menopause 6 (3): 233-241, 1999), reducing cholesterol (Potter et al., Am J Clin Nutr 68(6 suppl): 1375S-1379S, 1998), anti-cancer effect (Messina et al., Nutr Cancer 21(2): 113-131, 1994). Particularly, it is reported that genistein suppresses the activity of protein tyrosine kinase in a cell to intercept various growth factor signal, and suppresses isomerase to suppress the increase of cells directly (Murkies et al, J Clin Endocrinol Metab 83(2): 297-303, 1998). Anti-oxidative effects of isoflavone have been proved by various in vivo or in vitro experiments; for example, suppressing the activity of lipoxygenase, suppressing the generation of hydrogen peroxide or superoxide anion, and increasing the activity of anti-oxidative enzymes such as catalase, superoxide dismutase, glutathion peroxidase, glutathion reductase (Cai & Wei, Nutri Cancer 25(1): 1-7, 1996).

The composition of the present invention comprises genistein 0.001˜30 wt % to the total weight of the composition.

In addition, the composition of the present invention for treating obesity may contain other isoflavones that show similar effects with genistein. Such isoflavones are, for example, diadzein or glycitein.

The carnitine acts in the oxidation of fat is a generic name of β-hydroxy-γ-trimethylammonium butyric acid, and acts important role when transferring long-chain fatty acids with more than ten (10) carbon chains into the inner matrix of mitochondria from the outer membrane, and is represented by the following formula 2.

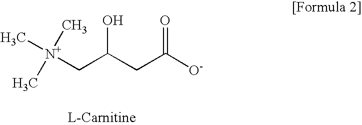

As seen above, L-cartinitine is an important component to generate energy by oxidating fats, which is prepared liver or kidney and contained generally in meat. When L-carnitine is not sufficient, concentration of fatty acid in the mitochondria becomes low, and as a result generation of energy also becomes low. In addition, it is also reported that CPT-1 using L-carnitine as substrate works as rate-limiting enzyme in the oxidation of fatty acid (Eaton, Prog Lipid Res 41(3): 197-269, 2002).

The composition of the present invention comprises L-camitine 0.001˜50 wt % to the total weight of the composition.

In summary, the present invention provides a composition comprising genistein represented by formula 1 and L-carnitine represented by formula 2 for treating obesity, which accelerates the oxidation of fat in a fat cell and can be administered orally.

The composition of the present invention may further comprises other components generally used in the field. The composition may be used as health food or medicines formulated as tablets, capsules, soft capsules, beads, granules, drinks, diet bars, chocolates, caramel, snacks, or the like.

### BRIEF DESCRIPTION OF THE DRAWINGS

FIG. 1 shows the effects of genistein and L-carnitine in the oxidation of fat in fat cells of male SD rat.

FIG. 2a is northern blotting test showing the acceleration of CPT-1 expression in liver by using the composition of the present invention.

A: high fat diet,

B: high fat diet+L-carnitine (0.2%),

C: high fat diet+genistein (0.2%),

D: high fat diet+L-carnitine (0.2%)+genistein (0.2%),

E: high fat diet+genistein (0.4%),

FIG. 2b is a graph of densitometry showing the acceleration of CPT-1 expression in liver by using the composition of the present invention.

HFD: high fat diet,

CA: L-carnitine

GE; genisteine

### PREFERRED EMBODIMENTS OF THE PRESENT INVENTION

Hereinafter, the present invention is described in more detail with Experimental Examples and Formulation Examples, however the scope of the present invention dose not restricted by the Examples. These Experimental Examples and Formulation Examples are described in order to explain the present invention, and it is clear for them skilled in the art that the scope of the present invention dose not restricted by the Examples. In the Experimental Examples and Formulation Examples, the unit of amount is wt %.

### REFERENCE EXAMPLE 1

Epididymal adipose tissues obtained from male SD rat were cut to small pieces, and 0.1% of collagenase (in DMEM without phenol red) was added then cultured for 2 hours at 37° C., and then filtered to obtain adipocyte.

### EXPERIMENTAL EXAMPLE 1

### Acceleration of Oxidation of Neutral Fat in Adipocytes of Male SD Rat

In order to verify acceleration of oxidation of neutral fat in adipocytes of male SD rat, experiment was performed using the adipocytes obtained in the Reference Example 1. A control was cultured in a medium not containing the composition of the present invention (experimental material). In the experimental samples, genistein and/or L-carnitine was added in 10 μmol unit. Results are calculated relatively converting the control to be 100%. Degrees of oxidation of fats are determined by measuring the concentration of glycerol separated into the medium from the adipocytes.

Experiments were performed by taking cell cultures prepared by adding colorless DMEM (Dulbeco's modified eagles medium) containing 0.5% bovine serum albumin (BAS) without fatty acid to the adipocytes. The amount of glycerol was measured with color reaction method using GPO-trinder kit purchased from Sigma (St. Louis, Mo., U.S.A) and absorption in 540 nm was measured with ELISA reader.

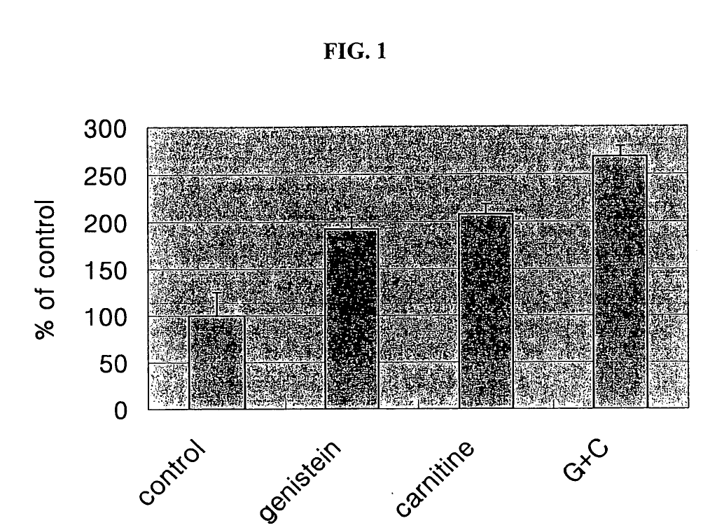

As seen in FIG. 1, when genistein and L-camitine were independently treated, oxidation of fatty acids increased 1.92 times and 2.07 times respectively compared with that of the control, and when genistein and L-camitine was treated together, oxidation of fatty acids increased 2.7 times.

### EXPERIMENTAL EXAMPLE 2

In order to verify the effects of the present composition to the metabolism of lipid in the fat animals induced by dieting high fat diet, Sprague-Dawley white male rat model was selected. In order to verify the effects of genistein, diadzein and glycitein to the obesity induced from high fat diet, rats of 6 weeks old were adapted for a week and assigned 12 rats for each experimental group. Experimental groups are as follows: (1) normal fat diet; (2) high fat diet; (3) high fat diet+genistein 0.2%; (4) high fat diet+L-camitine 0.2%; (5) high fat diet+genistein 0.2%+L-carnitine 0.2%; (6) high fat diet+diadzein 0.2%; (7) high fat diet+diadzein 0.2%+L-carnitine 0.2%; (8) high fat diet+glycitein 0.2%; and (9) high fat diet+glycitein 0.2%+L-carnitine 0.2%, and the rats were dieted for 8 weeks. Basic experimental diet was purified AIN-93G diet, and high fat diet was prepared to have fat occupy 36% of the total energy (18% of the total diet), normal fat diet was prepared to have fat occupy 17% of the total energy (7% of the total diet).

TABLE 1
Constitution of diet (g/kg diet)

| | | | high fat diet + | high fat | high fat diet + |
|---|---|---|---|---|---|
| | Normal diet | | genistein | diet + L- | genistein (0.2%) + L- |
| Group | 1) | high fat diet | (0.2%) | carnitine (0.2%) | carnitine (0.2%) |
| Corn powder | 529.486 | 419.486 | 417.486 | 417.486 | 415.486 |
| Casein | 200.0 | 200.0 | 200.0 | 200.0 | 200.0 |
| Sucrose | 100.0 | 100.0 | 100.0 | 100.0 | 100.0 |
| Soy bean oil | 70.0 | 180.0 | 180.0 | 180.0 | 180.0 |
| Genistein | — | — | 2.0 | — | 2.0 |
| Diadzein | — | — | — | — | — |
| Glycitein | — | — | — | — | — |
| L-carnitine | — | — | — | 2.0 | 2.0 |
| Fiber | 50.0 | 50.0 | 50.0 | 50.0 | 50.0 |
| Mineral | 35.0 | 35.0 | 35.0 | 35.0 | 35.0 |
| mixture 2) | | | | | |
| Vitamin | 10.0 | 10.0 | 10.0 | 10.0 | 10.0 |
| mixture 3) | | | | | |
| L-cysteine | 3.0 | 3.0 | 3.0 | 3.0 | 3.0 |
| Choline | 2.5 | 2.5 | 2.5 | 2.5 | 2.5 |
| vitartalate | | | | | |
| Tert-butyl | 0.014 | 0.014 | 0.014 | 0.014 | 0.014 |
| hydroquinone | | | | | |
| Total energy | 3498 | 4502 | 4498 | 4498 | 4494 |
| (kcal) | | | | | |

| | high fat diet + | high fat diet + | | high fat diet + |
|---|---|---|---|---|
| | diadzein | diadzein (0.2%) + | high fat diet + | glycitein (0.2%) + |
| group | (0.2%) | L-carnitine (0.2%) | glycitein (0.2%) | L-carnitine (0.2%) |
| Corn powder | 417.486 | 415.486 | 417.486 | 417.486 |
| Casein | 200.0 | 200.0 | 200.0 | 200.0 |
| Sucrose | 100.0 | 100.0 | 100.0 | 100.0 |
| Soy bean oil | 180.0 | 180.0 | 180.0 | 180.0 |
| Genistein | — | — | — | — |
| Diadzein | 2.0 | 2.0 | — | — |
| Glycitein | — | — | 2.0 | 2.0 |
| L-carnitine | — | 2.0 | — | 2.0 |
| Fiber | 50.0 | 50.0 | 50.0 | 50.0 |
| Mineral | 35.0 | 35.0 | 35.0 | 35.0 |
| mixture 2) | | | | |
| Vitamin | 10.0 | 10.0 | 10.0 | 10.0 |
| mixture 3) | | | | |
| L-cysteine | 3.0 | 3.0 | 3.0 | 3.0 |
| Choline | 2.5 | 2.5 | 2.5 | 2.5 |
| vitartalate | | | | |
| Tert-butyl | 0.014 | 0.014 | 0.014 | 0.014 |
| hydroquinone | | | | |
| Total energy | 4498 | 4494 | 4498 | 4494 |
| (kcal) | | | | |

1) Normal diet: AIN-93G diet
2) Mineral mixture: AIN-93G mineral mixture (g/kg mix)
3) Vitamin mixture: AIN-93G vitamin mixture (g/kg mix)

The amount of diet consumed and body weight was measure three times per week during dieting. After dieting, final body weight was measured and change of body weight are shown in table 2.

TABLE 2

| | | | Amount |
|---|---|---|---|
| | | | of food |
| | Before | After | consumed |
| | dieting (g) | dieting (g) | (g/day) |
| Normal diet | 176.1 ± 11.3 | 404.9 ± 24.4 | 27.1 ± 4.5 |
| (n = 12) | | | |
| high fat diet (n = 12) | 175.0 ± 15.4 | 480.1 ± 17.5 | 24.8 ± 7.6 |
| high fat diet + | 174.6 ± 22.5 | 433.5 ± 19.4 | 26.5 ± 3.8 |
| genistein (0.2%) | | | |
| (n = 12) | | | |
| high fat diet + | 173.9 ± 26.8 | 465.1 ± 31.8 | 25.7 ± 9.1 |
| L-carnitine (0.2%) | | | |
| (n = 12) | | | |
| high fat diet + | 175.9 ± 16.8 | 411.1 ± 23.3 | 25.8 ± 6.1 |
| genistein (0.2%) + | | | |
| L-carnitine (0.2%) | | | |
| (n = 12) | | | |
| high fat diet + | 174.3 ± 10.2 | 441.3 ± 19.4 | 26.8 ± 4.1 |
| diadzein (0.2%) | | | |
| (n = 12) | | | |
| high fat diet + | 172.9 ± 11.3 | 420.4 ± 12.8 | 24.5 ± 6.4 |
| diadzein (0.2%) + | | | |
| L-carnitine (0.2%) | | | |
| (n = 12) | | | |
| high fat diet + glycitein (0.2%) | 170.6 ± 9.4 | 446.4 ± 21.6 | 26.7 ± 4.7 |
| (n = 12) | | | |
| high fat diet + | 176.7 ± 13.7 | 424.8 ± 19.4 | 25.1 ± 2.4 |
| glycitein (0.2%) + | | | |
| L-carnitine (0.2%) | | | |
| (n = 12) | | | |

As shown in table 2, body weights of rats are not different each other before dieting. However, increase of body weight was small in the groups dieting genistein compared with those of control group with high fat diet. In addition the increase of body weight was much smaller in the groups dieting genistein together L-carnitine. However, when L-carnitine alone was dieted, body weight was almost the same with those of control group with high fat diet. In addition, increases of body weight were also small in the groups dieting diadzein and glycitein instead of genistein compared with those of control group. In conclusion, when genistein was dieted to the rat having obesity induced by high fat diet, increase of body weight was suppressed; and the effect of suppressing the increase of body weight was more significant when L-carnitine was dieted together. For reference, the amount of diet consumed was almost same through the testing groups.

### EXPERIMENTAL EXAMPLE 3

After 8 weeks of dieting with the groups of normal fat diet, high fat diet, high fat diet+genistein (0.2%), high fat diet+L-carnitine (0.2%), high fat diet+genistein (0.2%)+L-carnitine (0.2%), high fat diet+diadzein (0.2%), high fat diet+diadzein (0.2%)+L-camitine (0.2%), high fat diet+glycitein (0.2%) and high fat diet+glycitein (0.2%)+L-carnitine (0.2%), the rats were sacrificed to obtain epididymal adipose tissues. The above obtained epididymal adipose tissues were washed with saline and moistures were removed on a filter bed, then weights were measured.

TABLE 3

| | Weight of |
|---|---|
| | epididymal adipose (g) |
| Normal diet (n = 12) | 4.2 ± 0.31 |
| high fat diet (n = 12) | 8.4 ± 0.28 |
| high fat diet + genistein (0.2%) | 5.2 ± 0.67 |
| (n = 12) | |
| high fat diet + L-carnitine (0.2%) | 7.9 ± 0.45 |
| (n = 12) | |
| high fat diet + genistein (0.2%) + | 4.5 ± 0.71 |
| L-carnitine (0.2%) | |
| (n = 12) | |
| high fat diet + diadzein (0.2%) | 6.1 ± 0.35 |
| (n = 12) | |
| high fat diet + diadzein (0.2%) + | 4.8 ± 0.19 |
| L-carnitine (0.2%) | |
| (n = 12) | |
| high fat diet + glycitein (0.2%) | 6.3 ± 0.41 |
| (n = 12) | |
| high fat diet + glycitein (0.2%) + | 4.9 ± 0.13 |
| L-carnitine (0.2%) | |
| (n = 12) | |

As shown in the above table, the weight of epididymal adipose was small in the groups dieting genistein compared with those of control group with high fat diet, and the weight was much smaller when L-carnitine was dieted together. In conclusion, when genistein was dieted to the rats having obesity induced by high fat diet, increase of body adipose was suppressed; and the effect of suppressing the increase of body adipose was more significant when L-carnitine was dieted together. However, when L-carnitine alone was dieted, weight of epididymal adipose was almost the same with that of control group with high fat diet. In addition the weight epididymal adipose were also small in the groups dieting diadzein and glycitein instead of genistein compared with that of control group.

### EXPERIMENTAL EXAMPLE 4

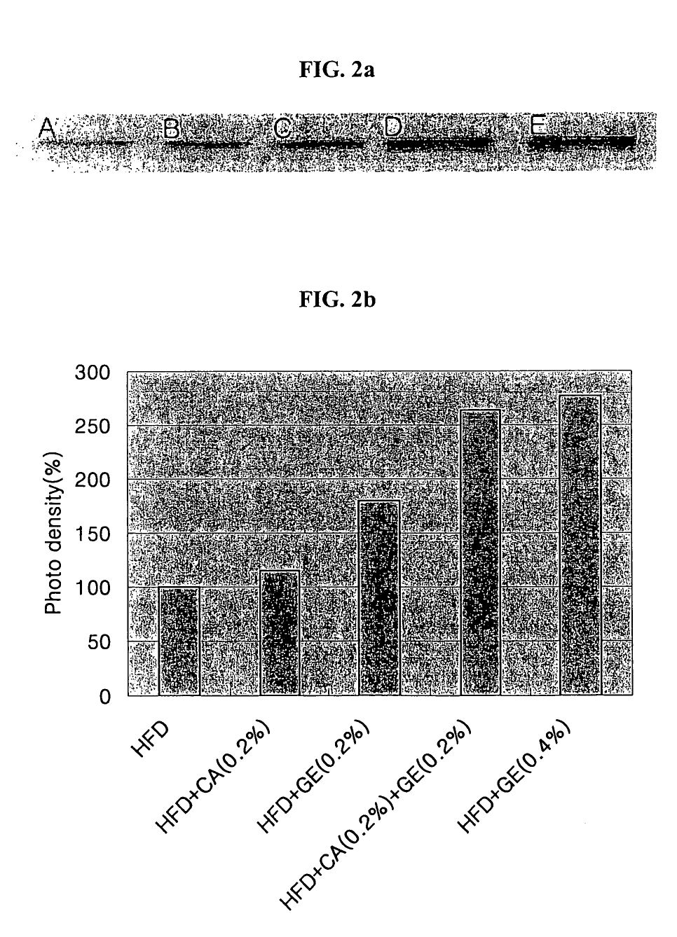

After 8 weeks of dieting with following four (4) groups of high fat diet, high fat diet+genistein (0.2%), high fat diet+genistein (0.2%)+L-camitine (0.2%) and high fat diet+genistein (0.4%), the rats were sacrificed to obtain liver tissues thereof. After homogenizing the liver tissues, RNA were extracted from the tissues using TRIZOL (Life Technologies, grand Island, N.Y., USA) consisting of phenol and guanidine isothiocyanate. Degree of expression of CPT-1 mRNA from the above extracted RNA was measured by northern blotting test, and expressed quantitatively by densitometry as shown in FIGS. 2a and 2b.

As shown in FIG. 2b, the expression of CPT-1 increased in the group dieting genistein compared with the group of high fat diet, and the increase was more significant when L-carnitine was dieted together. In addition, the expression of CPT-1 in the group of high fat diet+genistein (0.2%)+L-carnitine (0.2%) [HFD+CA(0.2%)+GE(0.2%)] was similar with that of the group of high fat diet+genistein (0.4%) [HFD+GE(0.4%)]. From the above result, it was verified that the expression of CPT-1 increases positively according to the concentration of genistein and that synergic effect is obtained when L-carnitine was dieted together. The above synergic effect can prevent such side effects that are caused when an excessive quantity of genistein was used, for example, disorder of sectretion in female rats or reduction of spermatozoa and sperm in male rats (Kazushi Okazaki et al., Arch Toxicol 2002, 76: 553-559; K. Barry Delclos et al., Reproductive toxicology 2001, 15: 647-663), in addition, which is very economic because the amount of genistein, very expensive material, used can be reduced.

### FORMULATION EXAMPLE 1

### Soft Capsules

80 mg of genistein, 180 mg of soybean oil, 2 mg of palm oil, 8 mg of vegetable oil, 4 mg of Cera Flava and 6 mg of lecithin were mixed, and 400 mg of the mixture was filled into each capsule according to conventional method to make a soft capsule.

### FORMULATION EXAMPLE 2

### Tablet

74 mg of genistein, 120 mg of L-carnitine, 200 mg of galacto-oligosaccharide, 60 mg of lactose and 140 mg of maltose were mixed, and granulated with fluid-bed dryer, then 6 mg of sugar ester was added thereto and made tablet with punching. The amount of final tablet was 600 mg.

### FORMULATION EXAMPLE 3

### Granules

80 mg of genistein, 120 mg of L-carnitine, 250 mg of anhydride crystalline glucose and 550 mg of starch were mixed and granulated using fluid-bed granulator, then packed. The amount of final granule was 1 g.

### FORMULATION EXAMPLE 4

### Drinks

80 mg of genistein, 120 mg of L-camitine, 10 g of glucose, 0.6 g of citric acid and 25 g of liquid oligosaccharide were mixed and 300 ml of purified water was added thereto, and separately filled into 200 ml bottles. After filling, the bottle was sterilized for 4˜5 seconds at 130° C. to obtain final drink.

### FORMULATION EXAMPLE 5

### Caramel

80 mg of genistein, 120 mg of L-carnitine, 1.8 g of corn syrup, 0.5 g of powdered nonfat milk, 0.5 g of soybean lecithin, 0.6 g of butter, 0.4 g of vegetable oil, 1.4 g of sugar, 0.58 g of margarine and 20 mg of salt were mixed and formed to a caramel. The amount of final caramel was 6 g.

### FORMULATION EXAMPLE 6

### Diet Bar

80 mg of genistein, 120 mg of L-camitine, 20 g of starch, 9 g of wheat flour, 11 g starch syrup, 11.6 g of maltose, 6 g of margarine, 30 mg of salt, 30 mg of citric acid, 140 mg of sodium carbonate and 2 g of sugar ester were mixed and formed to a bar. The amount of final diet bar was 60 g.

As described above, the composition of the present invention for diet and for preventing or treating obesity prevents and controls obesity, which comprises genistein that accelerates the expression of the Carnitine Palmitoyl Transferase-1 (CPT-1), an important enzyme in the pathway for the lipolysis of fatty acid, and L-carnitine that transports fatty acid into a mitochondria and accelerates oxidation of fat.

1. A composition for treating obesity comprising at least one component that accelerates the expression of Carnitine Palmitoyl Transferase-1 (CPT-1).

2. The composition for treating obesity according to claim 1, which accelerates oxidation (lipolysis) of neutral fat in adipocyte.

3. The composition for treating obesity according to claim 1, wherein the component that accelerates the expression of Carnitine Palmitoyl Transferase-1 (CPT-1) is genistein.

4. The composition for treating obesity according to claim 3, wherein the amount of genistein is 0.001-30 wt % based on the total weight of the composition.

5. The composition for treating obesity according to claim 3, which comprises diadzein or glycitein instead of genistein.

6. The composition for treating obesity according to claim 1, which has a formulation selected from the group consisting of tablet, capsule, soft capsule, bead, granule, drink, diet bar, chocolate, caramel and snack.

7. The composition for treating obesity according to claim 1, further comprising L-carnitine.

8. The composition for treating obesity according to claim 7, wherein the amount of L-carnitine is 0.001-50 wt % based on the total weight of the composition.
